(19) United States
(12) Patent Application Publication
(10) Pub. No.: US 2007/0239281 A1
(43) Pub. Date: Oct. 11, 2007

(54) FEMUR HEAD CENTER LOCALIZATION

(75) Inventors: Hubert Gotte, Munchen (DE); Martin Immerz, Grafelfing (DE)

Correspondence Address: DON W. BULSON (BrainLAB), RENNER, OTTO, BOISSELLE & SKLAR, LLP, 1621 EUCLID AVENUE - 19TH FLOOR, CLEVELAND, OH, 44115, US

(73) Assignee: BRAINLAB AB, Feldkirchen (DE)

(21) Appl. No.: 11/621,674

(22) Filed: Jan. 10, 2007

Related U.S. Application Data
(60) Provisional application No. 60/765,043, filed on Feb. 3, 2006.

(30) Foreign Application Priority Data
Jan. 10, 2006 (EP) 06 000 385 2

(51) Int. Cl. A61F 2/38; A61B 5/05

(52) U.S. Cl. 623/20.27; 600/424

(57) ABSTRACT

A method for localizing a femur head center of a knee using only a marker array attached to a tibia, wherein the knee is modeled as a joint having at least one degree of freedom includes: using a geometrical model to describe kinematical behavior of the joint, said geometrical model including joint elements and a geometrical description of a position and orientation of the joint elements; acquiring a range of motion of the tibia with a tracking system, wherein the femur head center is fixed relative to the tibia; calculating positions and orientations of the geometrical model to fit the acquired range of motion; and calculating a location of the femur head center from the calculated positions and/or orientations.

### RELATED APPLICATION DATA

This application claims priority of U.S. Provisional Application No. 60/765,043 filed on Feb. 3, 2006, which is incorporated herein by reference in its entirety.

### FIELD OF THE INVENTION

The present invention relates to a method and apparatus for determining a femur head center location without using a femur marker array.

### BACKGROUND OF THE INVENTION

When surgical procedures at the knee are conducted, a femur marker array and a tibia marker array typically are used to determine a position of the femur, particularly the femur head center and the tibia.

WO 2005/053559 A1 discloses an apparatus for providing a navigational array that can be used to track particular locations associated with various body parts such as a tibia and femur to which reference arrays are implanted. A position sensor can sense data relating to the position and orientation of the reference arrays in a prosthetic installation procedure, a surgeon can designate a center of rotation of a patient's femoral head for purposes of establishing the mechanical axis and other relevant constructs relating to the patient's femur according to which prosthetic components can ultimately be positioned. Such center of rotation can be established by articulating the femur within the acetabulum or a prosthesis to capture a number of samples of position and orientation information and thus in turn to allow the computer to calculate the average center of rotation.

### SUMMARY OF THE INVENTION

A location of the femur head center can be determined using only a tibia marker array (i.e., an array of markers), which also can be used for subsequent navigation purposes on the tibia or femur. A three-step approach including calibration, attachment and reproduction can be used to determine the femur head center.

Calibration

A kinematical model of a leg is shown in FIG. 1a, wherein femur center of rotation is determined using a tibia marker array TM. The tibia marker array TM is attached to the patient's leg L, and then, during the calibration procedure, the leg L is moved to different positions. The marker array TM can be either fixed directly to the tibia or can be fixed to the leg using other means, such as Velcro®, for example, without performing surgical steps to attach the marker array TM.

Attachment

The femur center of rotation position is virtually connected to the tibia marker array TM to describe its position for a specific user-defined position of the patient's leg, e.g., for a specific flexion as shown in FIG. 1b. This can be sufficient for navigated surgical steps on the tibia alone, as such surgical steps typically rely on the femur head position in a specific knee position or orientation, described below as “tibia-only workflow”. For example, a proximal tibia cut could be aligned to the femur mechanical axis established in 90 degree flexion of the knee joint.

Reproduction

After the patient has been moved, the previously determined center position can be transformed to camera space by reproducing the initial user-defined leg position and capturing corresponding tibia marker positions with the camera system (e.g., a tracking system), as shown in FIG. 2c.

Knee joint kinematics are simplified to a mechanical model with few (e.g., two or in a specific defined position of the tibia relative to the knee or the femur only one) fixed rotational degree of freedom. One possible concept is a model with two rotational degrees of freedom, as shown in FIG. 3a. A first hinge can be used to describe knee flexion and a second hinge can be used to describe tibia rotation within the knee joint KJ. The femur head center FHC sits at the end of a link attached to the flexion axis, while the tibia marker array TM sits at the end of a link attached to the rotation axis. These rotational axes form a simplified mechanical model of the knee joint KJ. Their positions and orientations with respect to each other and the marker array TM are the mechanical parameters of the model. In a simple example configuration, both rotational axes are orthogonal to one another and the femoral head center FHC moves on a regular sphere with respect to the tibia T, as shown in FIGS. 3a and 3b.

For a specific patient with a marker array TM attached to the tibia T in a specific position, the model parameters are unknown before calibration. After calibration they can be calculated.

Calibration

Calibration can be carried out with rotational and translational movements of the tibia T and the femur F around the femur head center FHC located in the pelvis, as shown in FIG. 1a. The center point itself is maintained in space while the leg is moved and the knee is bent during the calibration run.

The orientations and the locations of the two rotational axes of the knee joint hinges can be derived from a data set of positions of the tibia array acquired with the camera system. Furthermore, the location of the femur head center can be calculated with respect to the flexion hinge. With these parameters, the mechanical model is defined and can describe the possible locations of the femoral head center FHC in dependency to the current flexion and internal rotation angles applied to the hinges.

The calibration procedure utilizes the fact that the parameters of the model, except for the flexion and rotation angles, are the same for all acquired tibia positions during the calibration run. Furthermore, the femur head center position with respect to the camera coordinate system is constant during the tibia movements. If the mechanical model is applied to describe the possible femur head center points for all of the recorded tibia array positions, there is a common point in camera space contained by all of the models. This common point in camera space is the femur head center point FHC, as shown in FIG. 3d. The calibration algorithm varies the mechanical parameters to establish this common point with minimum error. Thus, a distance di (or “a” according to the Denavit-Hartenberg notation) of the femur head center FHC from the simplified knee joint KJ can be calculated so that a single point of intersection may be found. For distances larger or smaller than dᵢ there could be more points of intersection.

In general, the knee or one or more joint elements of a body can be modelled as a kinematical chain. This kinematical chain can be moved to determine parameters describing the model and to obtain the location of the center of rotation of one end element of the chain, e.g., an element of the kinematical chain that is fixed while using and tracking the movements of only a single marker or reference array connected to an opposite end element of the kinematical chain.

Biomechanical literature describing the behavior of the physiological knee joint support the idea of a hinge kinematic under certain circumstances. Hassenpflug J: “Gekoppelte Knieendoprothesen” describes in Der Orthopäde 6 (2003) 32, S. 484-489 that under external rotation, the orientation of the flexion axis remains fixed over a certain flexion range (mono-centric behavior). Thus, the knee joint degenerates to a single flexion hinge (external rotation stays fixed to a constant value), as shown in FIGS. 4a and 4b. Wetz H. et al.: “Die Bedeutung des dreidimensionalen Bewegungsablaufes des Femurotibialgelenks für die Ausrichtung von Knieführungsorthesen” in Der Orthopäde 4 (2001) 30, S. 196-207 supports the idea of simplifying knee kinematics to a flexion hinge in the flexion range of about 25 degrees to 90 degrees with his own findings on the location of the knee axes.

The reported physiological behavior can be used to further simplify the mechanical model by skipping the second hinge that is used for internal and external rotation, respectively (see, e.g., FIG. 3c). To achieve this, the tibia can be rotated to a specific location or position, such that further rotation of the tibia T is restricted or limited. Then, during further movement of the leg, the tibia is held in this location or position relative to the femur or knee. For maximum computing stability, it is preferred that calibration be conducted in the range of 30 degrees to 90 degrees flexion and concomitant maximum external respective internal rotation by the surgeon.

Attachment

After calibration, the femur head center location is defined within the kinematical model. Its position and orientation with respect to the tibia marker array TM is then computed for the user-defined current stance and virtually attached by means of a calculated transformation matrix to the tibia marker array TM (see, e.g., FIGS. 1b and 2b). This transformation is valid for the current stance. It can now be exploited for alignment purposes on the tibia, as described below in Example 1.

To enable later reproduction, the initial stance preferably is one with a mechanically reproducible femur center position with respect to the tibia (e.g., as full extension paired with high external rotation), as described below. Thus, it remains valid with respect to the tibia array despite any camera or patient movement.

Hassenpflug I. c. shows that the knee joint has a certain freedom for internal and external rotation, respectively, dependent on the current flexion angle (see FIGS. 4a and 4b). This freedom is minimized in full extension to a range of +/−8 degrees. Attachment, for example, can thus be carried out in full extension and maximum external rotation (8 degrees) to exploit this point of limit-stop as a reproducible stance. Given that no intermediate surgical steps have changed the kinematics of the joint, this stance can be reapplied at any time.

Reproduction

Surgical steps on the femur rely on the current femur head center position with respect to camera space. Before such a surgical step is navigated, the femur head center is reproduced in camera space (see FIG. 2c). After having positioned the leg in the reproducible stance, the position of the tibia marker array TM can be read by the camera system C and the known transformation matrix can be applied to calculate a current center position in camera space. As long as the patient's hip is not moved, the femoral head center FHC can be used for navigation. Since typical navigation steps, such as, for example, aligning a drill guide, can be carried out rather quickly, the hip center can be kept still for such short periods.

Thus, a femur marker array can be omitted to minimize trauma on the femur and to improve accessibility of the limited space within the knee joint during surgery, which is particularly useful for minimal invasive or time-critical surgical procedures. Avoiding a femur marker is highly valuable for minimal invasive surgical procedures such as uni-compartmental knee procedures, where a marker array on the femur cannot be attached because of limited space or time.

Although the precision of the described approach can be limited, e.g., by the quality of the mechanical knee model used for calibration, it is beneficial for procedures where less precision for the femur head is sufficient, and at the same time the application of a femoral marker array is not possible or desired. Such conditions apply to specific surgical procedures, e.g., for the Oxford uni-compartmental implant family due to its spherical constructions and the minimally invasive nature of the procedure.

### BRIEF DESCRIPTION OF THE DRAWINGS

The forgoing and other features of the invention are hereinafter discussed with reference to the drawings.

FIGS. 1a to 1c illustrate calibration, attachment and tibia navigation in an exemplary tibia-only procedure in accordance with the invention.

FIGS. 2a to 2d illustrate calibration, attachment, and reproduction after movement and femur navigation of an exemplary femur and tibia procedure in accordance with the invention.

FIGS. 3a to 3b illustrate an exemplary calculation of the femur head center in accordance with the invention.

FIGS. 4a and 4b illustrate exemplary rotational behavior of the knee joint according to Hassenpflug.

FIGS. 5a and 5b illustrate exemplary models of the knee having one and two degrees of freedom, respectively.

FIG. 6 is a block diagram of an exemplary computer system that can be used to carry out the method in accordance with invention.

### DETAILED DESCRIPTION

### Example I

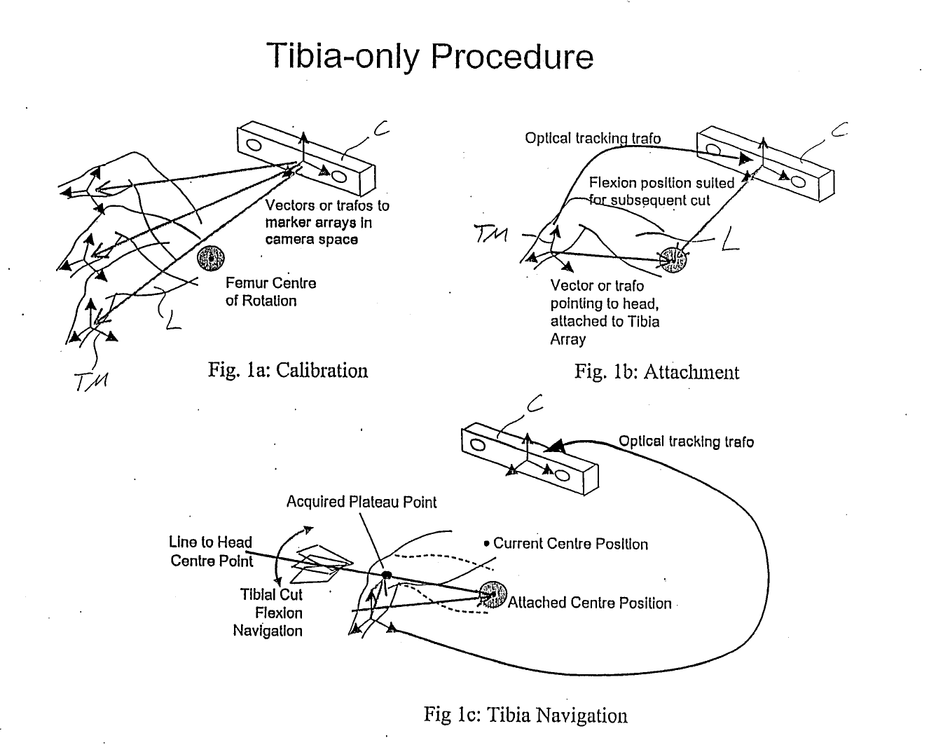

A tibia-only workflow for unicompartmental surgery is described with reference to FIGS. 1a-1c. Two tibial cuts can be applied without navigating any femur surgical steps, wherein the alignment of these tibial cuts depends on the position of the femur head center in 90 degree knee flexion. As described herein, this alignment can be achieved without using a femoral marker array and without time consuming femoral registration.

After moving the knee during the calibration step described herein, the calculated femur head center is “attached” to the tibia maker array in a fixed position, e.g., as a 90 degree flexion position, and relaxed external rotation state of the knee.

The flexion angle can be adjusted to 90 degrees before attaching the femur head center point. This can be supported by navigation without using a femoral marker array by simply connecting a line from the known femur head center point to the femoral notch. This point can be acquired with a pointer with the knee flexed in approximately 90 degree flexion, and is virtually attached to the tibia array, which is tracked on further movements. When the knee is brought in such a position (e.g., that the line from the femur head is orthogonal to the known tibia mechanical axis, the amount of flexion is nearly 90 degrees. In this state, the position of the femur head center defined in camera space is virtually attached to the tibia marker array, and tibia cuts are subsequently navigated.

This 90 degree flexion position is well suited for the subsequent vertical tibia cut, because it has to point to the femur head in 90 degree flexion of the knee. The cut can be subsequently navigated despite any simultaneous camera or patient movement, because the relevant femur center point is virtually attached to the tibia marker array.

### Example II

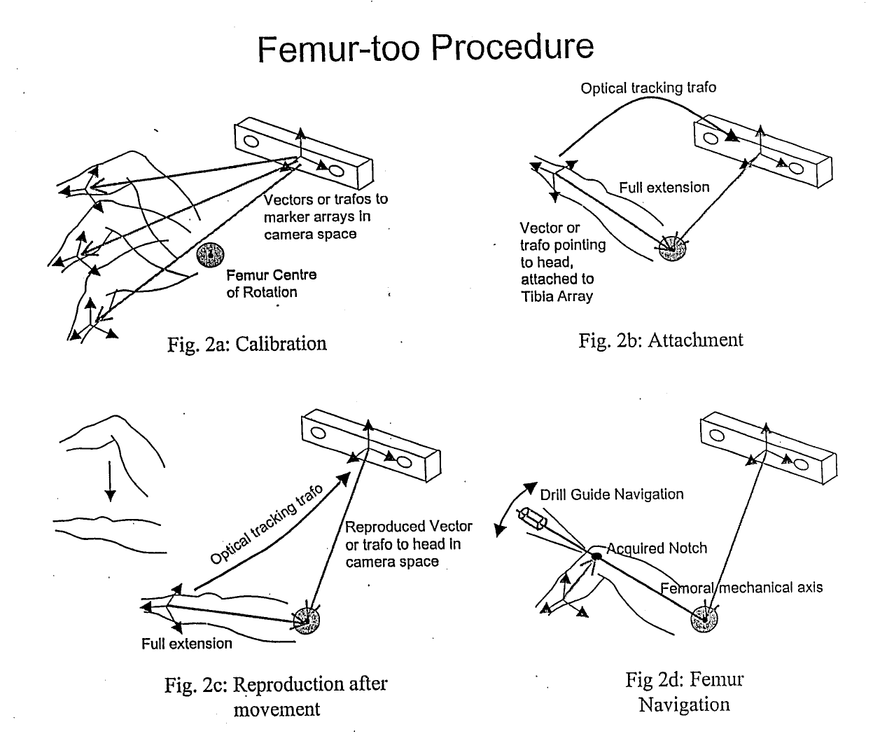

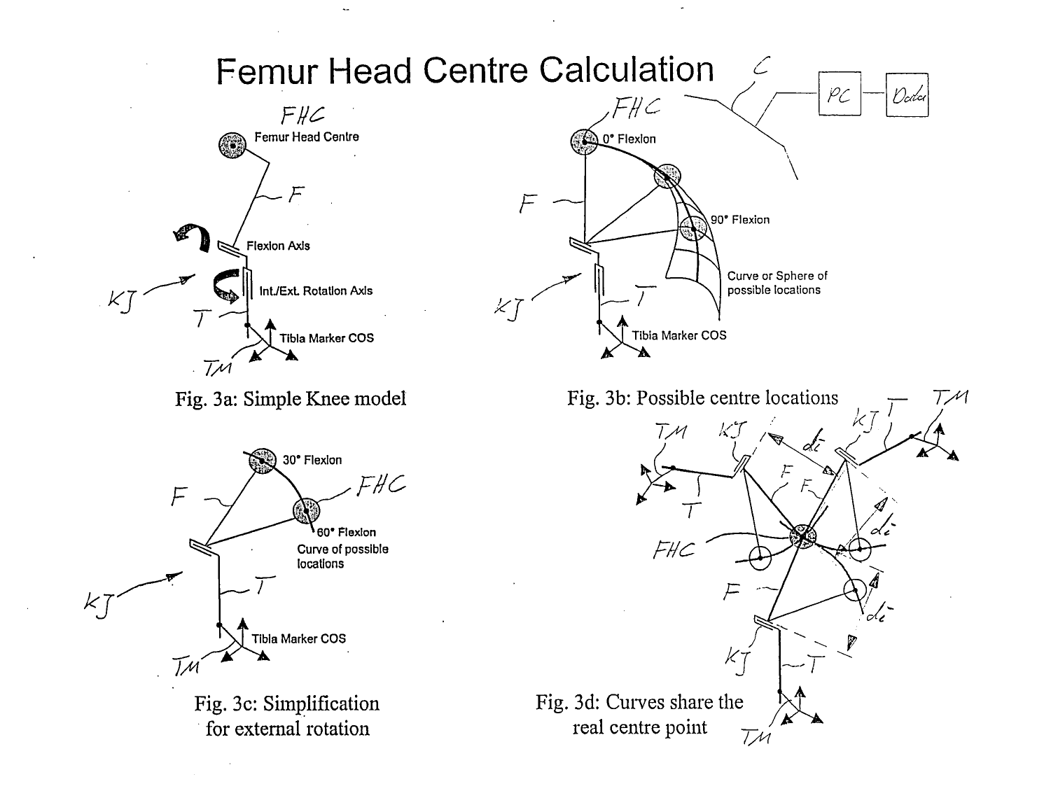

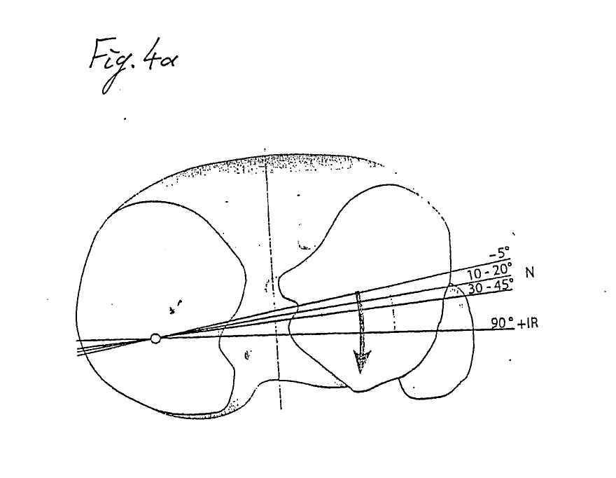

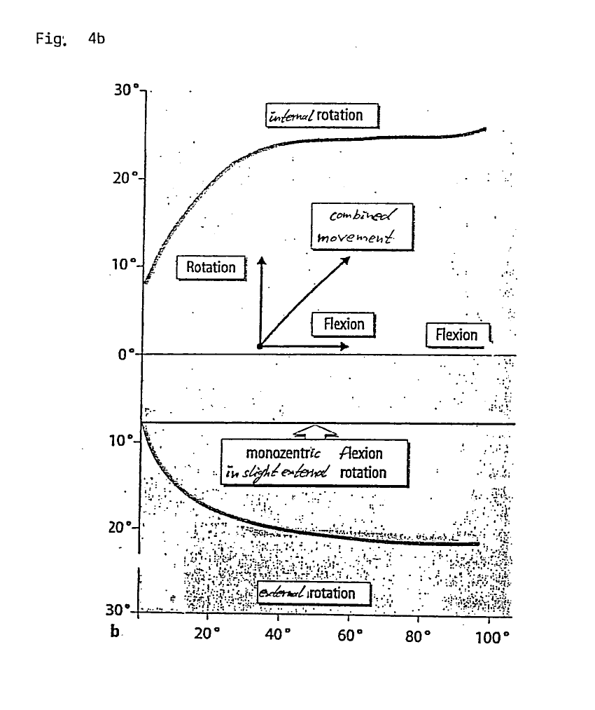

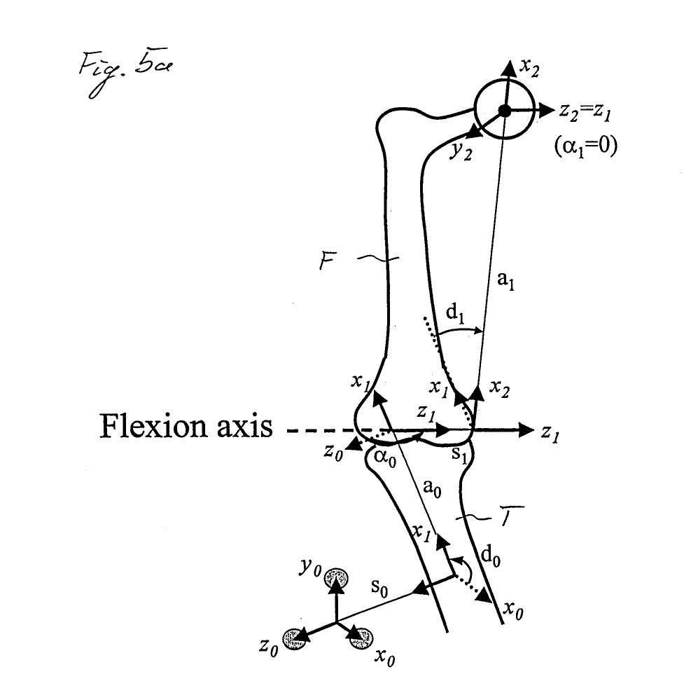

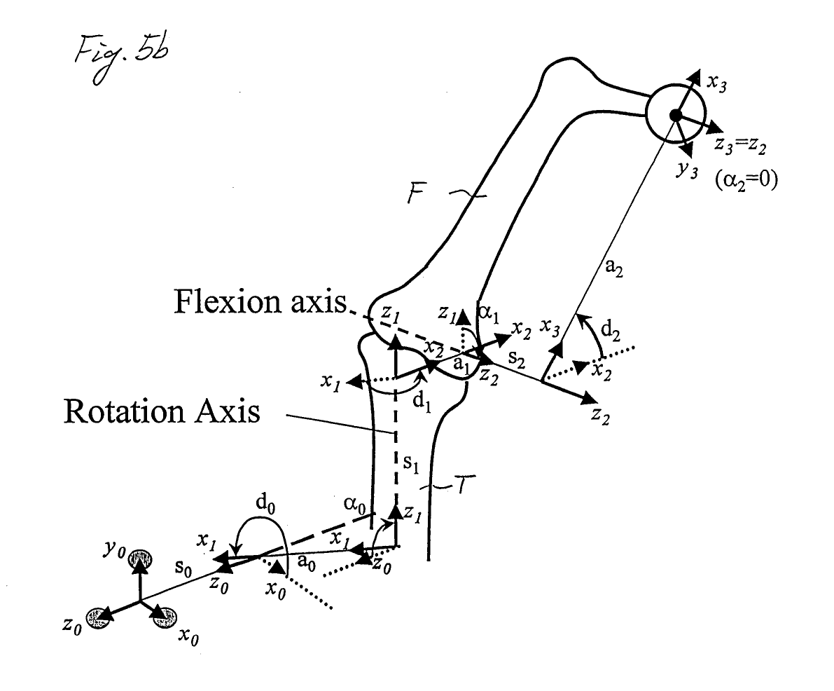

A femur and tibia workflow in Oxford unicompartmental surgery is described with reference to FIGS. 2a-2d. Besides tibia cuts, femur cuts also are performed in this example. A femoral drill guide can be navigated to geometrically define the location of the femur implant.

The rotational alignment of the drill guide can be defined in Varus-Valgus and in Flexion-Extension with respect to the femoral mechanical axis, which is defined by the femur head center point and a notch point on the proximal femur. As described herein, the drill guide alignment can be achieved without using a femoral marker array and without femoral registration.

The calculated femur head center is attached to the tibia marker array after calibration in full extension and maximum external rotation. This leg position is reproducible, because any rotational freedom of the knee is locked. From this point on, surgical steps causing movements of the patient or the leg may occur. Just before the drill guide is navigated, the full extension stance is re-applied to the knee by the surgeon and the tibia marker array is captured by the camera system. Then the femur head center position defined with respect to the tibia array can be transformed into camera space. Subsequent navigation of the drill guide can be done in camera space with respect to the known femur head center and the tracked tibia marker array. The leg can be brought into any convenient position for the drill guide navigation step as long as the femur head is kept in a fixed position relative to the tibia. Note, that unlike to the tibia-only-workflow described in Example I, any camera movement should be impeded during drill guide navigation.

FIG. 5a shows a model of a knee joint having one degree of freedom. A single or primitive joint element is a basic or elementary joint and can be described according to the notation of Denavit-Hartenberg by the parameters s, a, α and d, wherein s and a represent translations and α and d represent a rotation.

The reference array attached to the tibia T is represented by a coordinate system 0 with the axes x₀, y₀ and z₀. The parameters s₀, d₀, a₀, α₀, s₁, d₁, a₁ and α₁ describe the geometric model, wherein parameter d₁ represents the flexion of the knee joint.

The translation of the coordinate system 0 along its z-axis z₀ by the amount of s₀, the subsequent rotation around z₀ by d₀, the subsequent translation by a₀ along the now rotated x-axis and the subsequent rotation around the rotated x-axis by α₀ yields coordinate system 1 with the coordinate axes x₁, y₁ and z₁.

Translation of coordinate system 1 along z₁ by amount s₁, subsequent rotation around z₁ by d₁, subsequent translation by a, along the now rotated x-axis, subsequent rotation around the rotated x-axis by a₁ yields coordinate system 2 with the axes x₂, y₂, z₂. The origin of coordinate system 2 sits in the center of rotation inside the femur head.

The acquisition of marker positions is a prerequisite of determining the model parameters and can be performed as follows:
1. Extend the knee fully and apply maximum internal or external rotation so as to lock rotation of the knee. With the tibia reference array attached, circular movements around the femur center of rotation can be conducted.
2. Allow flexion in the knee joint up to 30 degrees to 40 degrees and repeat step 1 several times with changed flexion.
3. Vary adduction relative to abduction in the hip joint and repeat step 2 several times with changed adduction respectively abduction. Always keep the rotation of the knee joint locked.

FIG. 5b shows a model of the knee having two degrees of freedom. As for FIG. 5a, the reference array attached to the tibia is represented by a coordinate system 0 with the axes x₀, y₀ and z₀.

The translation of coordinate system 0 along its z-axis z₀ by amount s₀, subsequent rotation around z₀, by d₀, subsequent translation by a₀ along the now rotated x-axis and subsequent rotation around the rotated x-axis by α₀ yields coordinate system 1 with the axes x₁, y₁ and z₁.

The translation of coordinate system 1 along z₁ by amount s₁, subsequent rotation around z₁ by d₁, subsequent translation by a₁ along the now rotated x-axis, and subsequent rotation around the rotated x-axis by α₁ yields coordinate system 2 with the axes x₂, y₂, and z₂.

The translation of coordinate system 2 along z₂ by amount s₂, subsequent rotation around z₂ by d₂, subsequent translation by a₂ along the now rotated x-axis, subsequent rotation around the rotated x-axis by α₂ yields coordinate system 3 with the axes x₃, y₃ and z₃.

The origin of coordinate system 3 sits in the center of rotation inside the femur head. The parameters s₀, d₀, a₀, a₀, s₁, d₁, a₁, α₁, s₂, d₂, a₂ and α₂ describe the geometric model. Parameter d₁ represents the internal respectively external rotation and parameter d₂ the flexion of the knee joint.

To model the complex behavior of the knee joint more adequately and in order to gain precision, further sets of s, d, a and α parameters may be introduced for further degrees of freedom.

The acquisition of marker positions as prerequisite to determining the model parameters can be performed as follows:
1. Extend the knee fully and apply maximum internal or external rotation so as to lock rotation of the knee. With the tibia reference array attached, circular movements around the femur center of rotation can be conducted.
2. Allow flexion in the knee joint up to 30 degrees to 40 degrees and repeat step 1 several times with changed flexion. Release the locked rotation and constantly change the rotation within its physiological range.
3. Vary adduction relative to abduction in the hip joint and repeat step 2 several times with changed adduction relative to abduction.

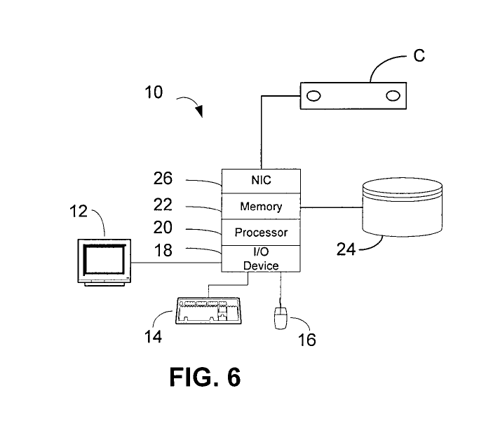

FIG. 6 illustrates the computer 10, which may be used to implement the method described herein, in further detail. The computer 10 may include a display 12 for viewing system information, and a keyboard 14 and pointing device 16 for data entry, screen navigation, etc. A computer mouse or other device that points to or otherwise identifies a location, action, etc., e.g., by a point and click method or some other method, are examples of a pointing device 16. The display 12, keyboard 14 and mouse 16 communicate with a processor via an input/output device 18, such as a video card and/or serial port (e.g., a USB port or the like).

A processor 20, such as an AMD Athlon 64® processor or an Intel Pentium IV® processor, combined with a memory 22 execute programs to perform various functions, such as data entry, numerical calculations, screen display, system setup, etc. The memory 22 may comprise several devices, including volatile and non-volatile memory components. Accordingly, the memory 22 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 20 and the memory 22 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database 24. The storage medium may be a hard drive, for example, or any other storage means that can retain data, including other magnetic and/or optical storage devices. A network interface card (NIC) 26 allows the computer 10 to communicate with other devices, such as the camera system C.

A person having ordinary skill in the art of computer programming and applications of programming for computer systems would be able in view of the description provided herein to program a computer system 6 to operate and to carry out the functions described herein. Accordingly, details as to the specific programming code have been omitted for the sake of brevity. Also, while software in the memory 22 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for localizing a femur head center of a knee using only a marker array attached to a tibia, wherein the knee is modeled as a joint having at least one degree of freedom, comprising:
using a geometrical model to describe kinematical behavior of the joint, said geometrical model including joint elements and a geometrical description of a position and orientation of the joint elements;
acquiring a range of motion of the tibia with a tracking system, wherein the femur head center is fixed relative to the tibia;
calculating positions and orientations of the geometrical model to fit the acquired range of motion; and
calculating a location of the femur head center from the calculated positions and/or orientations.

2. The method of claim 1, wherein the joint elements are primitive joint elements.

3. The method according to claim 1, wherein acquiring a range of motion includes bringing the tibia to a position that restricts at least one degree of movement of the knee joint such that the knee joint has only a single degree of freedom, and moving the femur and/or the tibia to move the knee.

4. The method according to claim 1, further comprising navigating the knee via the tibia marker array.

5. The method according to claim 1, further comprising moving the tibia, femur and/or knee to a fixed or reproducible flexing position to restrict at least one degree of movement of the knee.

6. The method according to claim 1, wherein calculating positions and orientations includes determining a position of the knee joint or of the joint elements of the knee joint relative to the tibia marker array.

7. A computer program embodied on a computer readable medium for localizing a femur head center of a knee using only a marker array attached to a tibia, wherein the knee is modeled as a joint having at least one degree of freedom, comprising:
code that use a geometrical model to describe kinematical behavior of the joint, said geometrical model including joint elements and a geometrical description of a position and orientation of the joint elements;
code that acquires a range of motion of the tibia with a tracking system, wherein the femur head center is fixed relative to the tibia; and
code that calculates positions and orientations of the geometrical model to fit the acquired range of motion;
code that calculates a location of the femur head center from the calculated positions and/or orientations.

8. A method for localizing a femur head center of a knee using only a marker array attached to a tibia, wherein the knee is modeled as a joint having at least one degree of freedom, comprising:
modeling the knee joint as a kinematical chain;
calculating a distance dᵢ such that lines of movement of a point having the distance dᵢ from the knee joint or from the joint element closest to the femur head center coincide in a single point, wherein the single point is considered as the femur head center.

9. The method according to claim 8, further comprising navigating the knee via the tibia marker array.

10. The method according to claim 8, further comprising moving the tibia, femur and/or knee to a fixed or reproducible flexing position to restrict at least one degree of movement of the knee.

11. A computer program embodied on a computer readable medium for localizing a femur head center of a knee using only a marker array attached to a tibia, wherein the knee is modeled as a joint having at least one degree of freedom, comprising:
code that models the knee joint as a kinematical chain;
code that calculates a distance dᵢ such that lines of movement of a point having the distance dᵢ from the knee joint or from the joint element closest to the femur head center coincide in a single point, wherein the single point is considered as the femur head center.

12. An apparatus for localizing the femur head center of a knee joint using only a tibia marker array connected to the tibia, comprising:
a camera for localizing the tibia marker array;
a processor and memory, said processor operatively coupled to the camera to obtain the positional data of the tibia marker array from camera images of the tibia marker array;
a database stored in memory and including a kinematic model of the knee joint, wherein the model has at least one degree of freedom; and
logic stored in memory and executable by the processor so as to calculate a distance dᵢ such that lines of movement of a point having a distance dᵢ from the knee joint or from a joint element closest to the femur head center in the kinematical model coincide in a single point, wherein the single point is considered to be the femur head center.
